(19) United States
(12) Patent Application Publication
(10) Pub. No.: US 2025/0318864 A1
(43) Pub. Date: Oct. 16, 2025

(54) COMBINATION HANDPIECE WITH PRESSURE ACTIVATED LOCK FOR USE WITH CRYOGENIC DEVICES

(71) Applicants: James Allen ALEXANDER, Excelsior, MN (US); Jonathon MCHALE, Cincinnati, OH (US); Eric MORRIS, Minneapolis, MN (US)

(72) Inventors: James Allen ALEXANDER, Excelsior, MN (US); Jonathon MCHALE, Cincinnati, OH (US); Eric MORRIS, Minneapolis, MN (US)

(21) Appl. No.: 18/657,402

(22) Filed: May 7, 2024

Related U.S. Application Data
(60) Provisional application No. 63/634,727, filed on Apr. 16, 2024.

(51) Int. Cl. A61B 18/02; A61B 18/00; A61B 90/90

(52) U.S. Cl. CPC A61B 18/02; A61B 90/90; A61B 2018/00178; A61B 2018/00577; A61B 2018/00821; A61B 2018/00988; A61B 2018/0262

(57) ABSTRACT

Disclosed herein are systems and methods for a cryogenic device for ablating tissue. The device can comprise a handle comprising one or more tabs on lateral sides of the handle. The handle can further comprise an unlocked configuration in which the one or more tabs are pressed into an interior of the handle. The device can further comprise an elongated probe extending from the handle. The device can further comprise a receptacle, wherein the handle connects the elongated probe to the receptacle. The device can further comprise a plate within the handle. The plate can be configured to lock the one or more tabs in a locked configuration of the handle such that the handle is locked into the receptacle when one or more fluid supply passageways is pressurized, wherein the plate is moved axially by one or more elastic members between the locked configuration and the unlocked configuration.

### FIELD OF TECHNOLOGY

The present disclosure relates generally to the field of cryogenic devices, and more specifically, handpiece elements for incorporating various features of cryogenic devices.

### BACKGROUND

Current handles for cryogenic devices require multiple connections in order to correctly function. Such connections include lines for pressure, exhaust, and thermocouple connections. Accordingly, this requires multiple connection points within the handle of the device. However, multiple connection points can result in confusion for the operator, which can result in lost time before the device is used.

Further, the confusion caused by multiple connections can result in the operator not knowing which connections can be removed safely when pressure levels within the device are at unsafe levels for disconnection.

Therefore, there remains a need for methods and devices that combine multiple connections for a cryogenic device into a single handpiece. Such methods and devices should provide a locking mechanism for the user to disconnect the handpiece when the device pressure is at a predetermined value.

### SUMMARY

A cryogenic device for ablating tissue is disclosed herein. The device can comprise a handle comprising one or more tabs on lateral sides of the handle. The handle can further comprise an unlocked configuration in which the one or more tabs are pressed into an interior of the handle. The device can further comprise an elongated probe extending from the handle, the elongated probe having a proximal end and a distal end, the distal end defining an end effector. The device can further comprise one or more fluid supply passageways extending within the elongated probe from the handle. The device can further comprise one or more elastic members around each of the one or more fluid supply passageways. The device can further comprise a receptacle, wherein the handle connects the elongated probe to the receptacle. The device can further comprise a plate within the handle. The plate can be configured to lock the one or more tabs in a locked configuration of the handle such that the handle is locked into the receptacle when the one or more fluid supply passageways is pressurized, wherein the plate is moved axially by the one or more elastic members between the locked configuration and the unlocked configuration.

The one or more fluid supply passageways comprises an exhaust passageway and an inlet passageway. The device can further comprise an identification card within the handle. The identification card can be a FRAM card. The identification card can be replaceable. The receptacle can be locked to the handle in the locked configuration. The receptacle can be locked to the handle in the locked configuration when the one or more elastic members coupled to the plate are in a compressed state. The receptacle can be removed from the handle when the one or more elastic members coupled to the plate are in a released state. The device can further comprise one or more electrical connections coupled to the handle, the electrical connections can each be connected to a thermocouple. The one or more tabs can release to the unlocked configuration when pressure within the one or more fluid supply passageways is reduced to a predetermined threshold. The device can further comprise one or more pressure sensors coupled to the handle.

Methods for ablating tissue with a cryogenic device are also disclosed herein. The method can comprise connecting a handle to a receptacle coupled to an elongated probe, wherein the handle comprises one or more tabs on lateral sides of the handle and a plate within an interior of the handle that is configured to slide axially. The method can further comprise pressurizing one or more fluid supply passageways extending within the elongated probe from the handle, wherein the plate slides axially into a locked configuration when the one or more fluid supply passageways are pressurized. The method can further comprise delivering fluid through the one or more fluid supply passageways to a distal end of the elongated probe. The method can further comprise ablating at least a portion of the tissue in contact with the distal end of the elongated probe. The method can further comprise removing the handle from the receptacle by pressing the one or more tabs into the interior of the handle.

### BRIEF DESCRIPTION OF THE DRAWINGS

FIG. 1 illustrates a view of the cryoprobe of the present disclosure showing the cryogenic device adapted for the supply and control of cryofluid.

FIG. 2 illustrates a vertical sectional view of the cryoprobe of FIG. 1 taken through the central line.

FIG. 3 illustrates a cross-sectional view of a malleable end effector.

FIG. 4 illustrates an enlarged cross-sectional view of the distal tip of the malleable end effector.

FIG. 5 illustrates a cross-sectional view of an alternate configuration of the distal tip of the malleable end effector.

FIG. 6 illustrates an exploded view of a handle for a cryogenic device.

FIGS. 7A and 7B illustrate perspective views of the handle for a cryogenic device.

FIGS. 8A and 8B illustrate cross-sectional views of the handle coupled to a receptacle.

FIG. 9 illustrates a side view of a cryogenic device for use with the handle.

### DETAILED DESCRIPTION

The exemplary embodiments of the present disclosure are described and illustrated below to encompass exemplary cryogenic devices and, more specifically, encompass cryogenic devices and methods of manufacturing the same, where the cryogenic devices can be used for surgical applications to deliver cooling to one or more tissue locations. In addition, the exemplary embodiments are directed to methods of using cryogenic devices as part of surgical procedures. Of course, it will be apparent to those of ordinary skill in the art that the embodiments discussed below are exemplary in nature and can be reconfigured without departing from the scope and spirit of the present invention. However, for clarity and precision, the exemplary embodiments as discussed below can include optional steps, methods, and features that one of ordinary skill should recognize as not being a requisite to fall within the scope of the present invention.

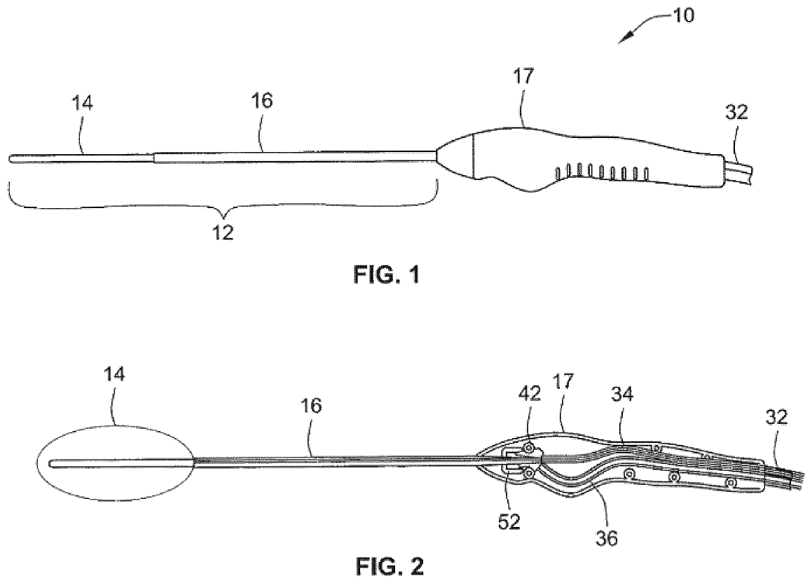

With reference to FIGS. 1 and 2, a first exemplary cryogenic device 10 includes an elongated probe 12 that terminates in a malleable end effector 14. In use, the malleable end effector generates surface temperatures below −40° C. When the end effector 14 is applied to the tissue to be treated, freezing of tissue coming into direct contact with the probe results. Surrounding tissue is sequentially frozen by the withdrawal of heat from the tissue as the probe maintains contact with tissue over time.

The disclosed device 10 may be used in an open procedure on an arrested heart, with the end effector 14 being applied to the endocardium or inner surface of the heart (through a purse-string opening), or alternatively to the epicardium or outer surface of the heart. The freezing of the cardiac tissue causes an inflammatory response (cryonecrosis) that blocks the conduction of electrical pulses.

The device 10 can comprise an elongated probe tube 15 whose distal portion comprises the malleable end effector 14. The tube has a smooth outer surface over its entire length. A semi-rigid sleeve 16, made of polycarbonate, overlies the proximal portion of the elongated probe tube, and both the tube and sleeve extend from and are secured to a handle 17, which is known in the prior art. In this exemplary embodiment, the device 10 has an overall length of approximately 43 cm, with the malleable end effector 14 having a variable length of up to approximately 10 cm, and the semi-rigid sleeve 16 and the handle 17 having a combined length of approximately 33 cm. If employed in a robotic device, the length of the probe tube may vary. All materials used in the device 10 that are exposed to the cryofluid may be compatible with the cryofluid used in the device, and components intended for patient contact may be biocompatible. The device (and its packaging) can also be gamma stable, as gamma sterilization is an exemplary sterilization method.

The end effector/probe tube 15 is constructed of a relatively soft metal, such as Series 1000 aluminum alloy. Alternatively, gold, gold alloys, stainless steel, nitinol, or other malleable metallic alloys that have suitable thermal conductivity may be used. In exemplary form, the end effector 14 is malleable and formed into various shapes appropriate for making the different ablation lines, but is stiff enough for tissue conformance and to maintain its shape when applied to cardiac tissue without any secondary reinforcement. Likewise, the exemplary end effector is capable of being bent in an arcuate manner to have a minimum radius of approximately 0.5 in.

The end effector 14 of the probe tube 15 is provided with internal flexible support walls to prevent kinking and to help maintain the circular cross-section of the end effector during deformation. In this exemplary embodiment, the end effector 14 is supported internally by a coiled spring 20 made of stainless steel. The spring 20 may also serve to capture segments of the end effector in the event that the end effector should fracture. The coiled spring 20 may be free-floating, or it may be retained in place on the interior of the end effector 14 by frictional engagement with the inner wall of the end effector, with at least a few coils of the spring being oversized to frictionally engage the inner wall of the end effector. In this exemplary embodiment, the spring 20 has a pitch of from about 0.018 in. to about 0.022 in. and an outside diameter of from about 0.115 in. to about 0.125 in.

As seen in FIG. 2, a multipart coupling comprising a gas exchanger fitting 42 and nut 52 can connect the probe tube to the cryofluid delivery and return lines 34, 36. The exchanger fitting 42, a ferrule, and nut 52, may each be made from stainless steel, and are operative to secure the delivery/return lines and probe tube together.

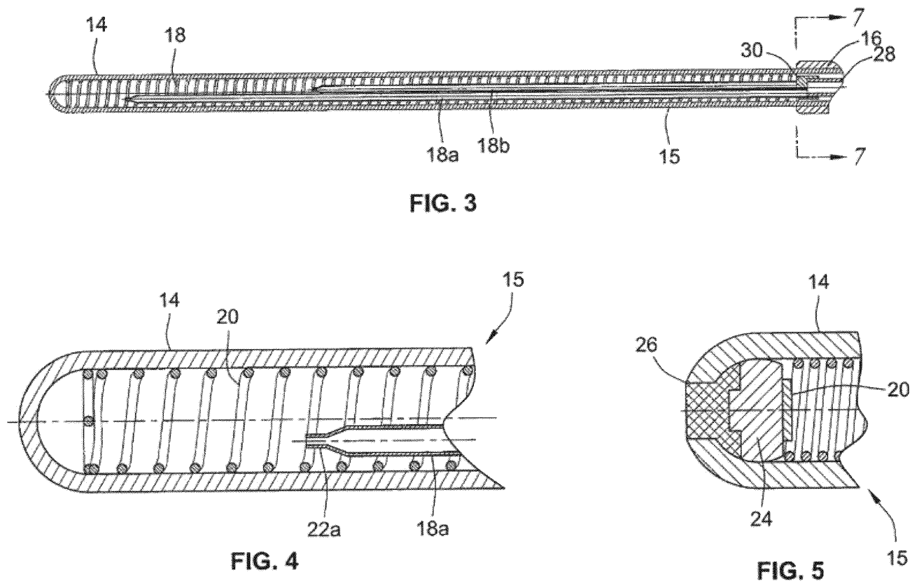

The end effector 14 has a smooth exterior surface for contacting the tissue to be ablated. With reference to FIGS. 3 and 4, it is seen that the distal tip of the end effector 14 is closed and forms a blunt, atraumatic, generally hemispherical shape. This can be accomplished by limiting the opening in the end effector, by casting, spin forming, etc.

As shown in FIG. 5, the blunt distal tip of the end effector may be formed by limiting the opening in the tube 15, and closing the remaining opening with a separate plug 24 that may be formed of aluminum. The plug may be held in place by an epoxy 26 or other suitable adhesive. Alternatively, the plug 24 may be soldered, welded, press fit or cast into position.

All surfaces of the cryoprobe that are not intended for patient contact may be insulated for the protection of both non-target tissue and the user. To this end, the interior of the sleeve 16 creates an air pocket that serves to insulate the portion of the probe tube proximal of the end effector 14, thus protecting adjacent non-treated tissue from freezing tissue that may come into contact with the exposed portion of the sleeve 16. Similarly, the handle provides an insulated surface to hold the probe tube in position while manipulating the end effector.

Inside the end effector 14 a Joule-Thomson Effect is formed where the cryofluid undergoes expansion. The Joule-Thomson Effect is created by the expansion of gas that occurs as the cryofluid moves through the small orifice from each of the high pressure supply tubes into the low pressure expansion chamber comprised by the probe tube. Temperatures within the probe tube can fall below −60° C., and provide for surface temperatures of the end effector to reach less than −45° C., when nitrous oxide gas is used as the cryofluid.

In the illustrated embodiment, the end effector 14 houses a plurality of separate gas delivery passageways in the form of malleable supply tubes or hypotubes (not necessarily limited to three in number and made of stainless steel in this exemplary embodiment) designated 18a, 18b and 18c. Each of the supply tubes 18a, 18b, and 18c terminates in a reduced orifice 22a, 22b and 22c that forms a nozzle to deliver the gas into the expansion chamber (probe tube). Each nozzle has a cross-sectional area that achieves a flow rate of 600-630 ccm at 15 psi. In practice, this results in the individual orifices having an inside diameter of from about 0.003 to about 0.010 in. and a corresponding cross sectional area of from about 0.00000707 sq. in. to about 0.0000785 sq. in. The orifices are staggered lengthwise at 0.7 to 0.9 in. (2 cm) intervals.

In order to deliver cryofluid to the device 10, a flexible tubeset 32 is provided that extends from the handle 17 and connects the probe tube to the console, the handle providing strain relief for the tubeset. The tubeset 32 comprises a high pressure (700 psi) delivery (inlet) line 34, preferably including a filter, that supplies cryofluid, such as nitrous oxide gas, to the cryoprobe and a low pressure (approximately 30 psi to 50 psi) return (exhaust) line 36 that evacuates the expanded cryofluid from the probe. The flexible delivery and return lines are capable of withstanding a minimum pressure of 1400 psi, with the delivery line having an inside diameter of 0.078 in., and the return line having a minimum inside diameter of 0.142 in.

In some variations, the devices and methods disclosed herein can be used in applications for delivering cryofluid for nerve blocks or cryoanalgesia.

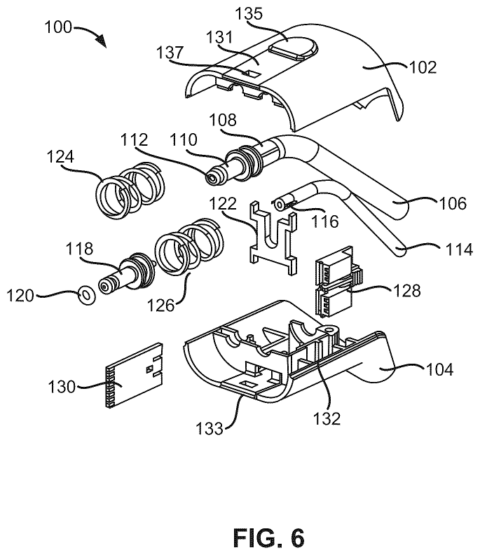

Referring to FIG. 6, an exemplary handle 100 according to the present invention is shown in an exploded view. The handle 100 can comprise a top shell 102 and a bottom shell 104 that are coupled to each other to form an exterior of the handle 100. The top shell 102 and the bottom shell 104 can both comprise embedded fittings and/or detents to hold various components within the handle, as will be described below.

The handle 100 can comprise an exhaust hose 106, a crimp collar 108, and an exhaust connector 110. The exhaust hose 106 can be placed within a slot between the top shell 102 and the bottom shell 104 when the shells are coupled together. The exhaust hose 106 can evacuate cryofluid from the probe 10 as desired. The crimp collar 108 can be positioned around the exhaust hose 106 to seal a connection between the exhaust hose 106 and the exhaust connector 110 at a distal end of the exhaust hose 106. An O-ring 112 can be provided at the end of the exhaust connector 110 to seal the system by preventing leaks.

An inlet hose 114 can be placed within a slot between the top shell 102 and the bottom shell 104 when the shells are coupled together. The inlet hose 114 can introduce cryofluid from a fluid source to the end effector 14 to ablate tissue as desired. The inlet hose 114 can comprise a crimp collar 116 positioned around the inlet hose 114 to seal a connection between the inlet hose 114 and an inlet connector 118 at a distal end of the inlet hose 114. An O-ring 120 can be provided at the end of the inlet connector 118 to seal the system by preventing leaks.

A plate 122 can be coupled to the exhaust hose 106 and the inlet hose 114 to lock the handle 100 between configurations. The plate 122 can rest within fittings between the top shell 102 and the bottom shell 104 and can move axially upon movement of elastic members coupled to the exhaust hose 106 and the inlet hose 114.

In some variations, the plate 122 can be coupled to both the exhaust hose 106 and inlet hose 114. In some variations, the plate 122 can be coupled to only one of the exhaust hose 106 and the inlet hose 114.

The plate can comprise one or more legs that can couple to the top shell 102 or the bottom shell 104 such that the plate 122 can slide axially in response to movement of springs 124, 126.

The handle 100 can further comprise a card socket 128 within the shells. The card socket 128 can be placed within a slot 132 between the top shell 102 and the bottom shell 104. The card socket can carry an identification card 130. The identification card 130 can be an EEPROM (Electrically Erasable Programmable Read-Only Memory) or FRAM (Ferroelectric Random Access Memory) card. In addition to providing identification of the handle 100 itself, the identification card 130 can also be used to store data for diagnostic purposes and can be replaceable accordingly.

In this variation, the handle 100 can comprise a top tab 131 and a bottom tab 133 that can be pressed inwardly by the user to release the locking mechanism within the handle 100. The tabs 131, 133 can comprise a button 135 that locks into a receptacle (not shown) that the handle 100 can couple to. The tabs 131, 133 can be positioned on the outer surfaces of top shell 102 and bottom shell 104, respectively. Button 135 is a raised surface such that the user can push button 135 into the top shell 102 of the handle 100. By pushing in button 135, an opening 137 in tab 131 is pushed outward thereby releasing from the receptacle. Tab 131 can act on a living hinge incorporated within such that pushing on the button 135 can pivot the tab 131 near or at the middle of tab 131 causing the opening 137 that secures onto the receptacle to move outward.

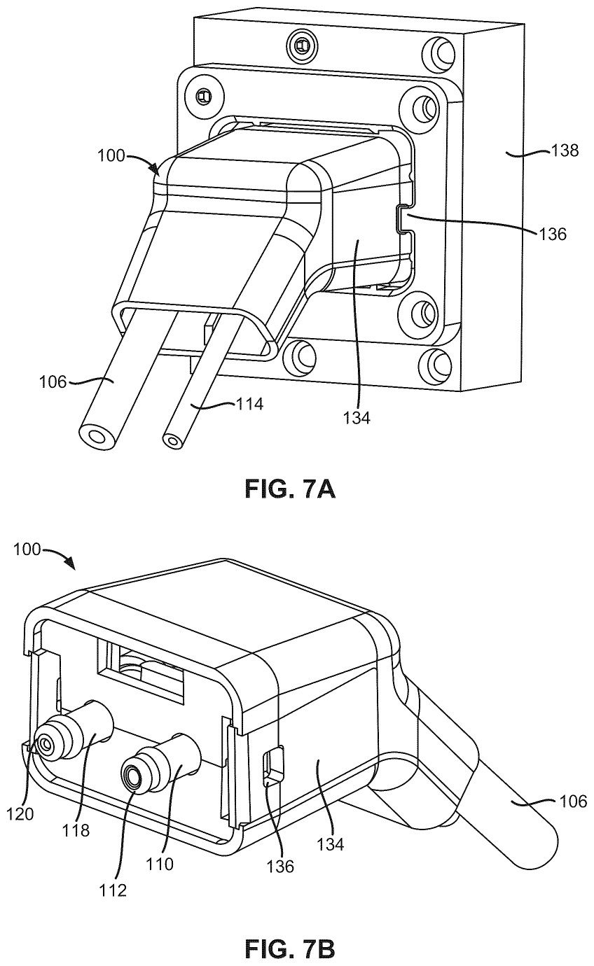

FIGS. 7A-B illustrate a variation of the handle 100 having tabs 134 on lateral sides of the handle 100. The tabs 134 can be pressed inwardly by the user to release the locking mechanism within the handle 100. The tabs 134 can comprise a detent 136 that locks into a receptacle 138 that the handle 100 can couple to. The tabs 134 can be angled or tapered laterally outward towards the distal end of the handle 100.

The inlet connector 118 and the exhaust connector 110 can connect to respective female connectors in the receptacle 138 to connect their respective hoses to lumens within the receptacle 138 and subsequently, lumens within the probe end effector 14 for introduction or exhaustion of cryofluid.

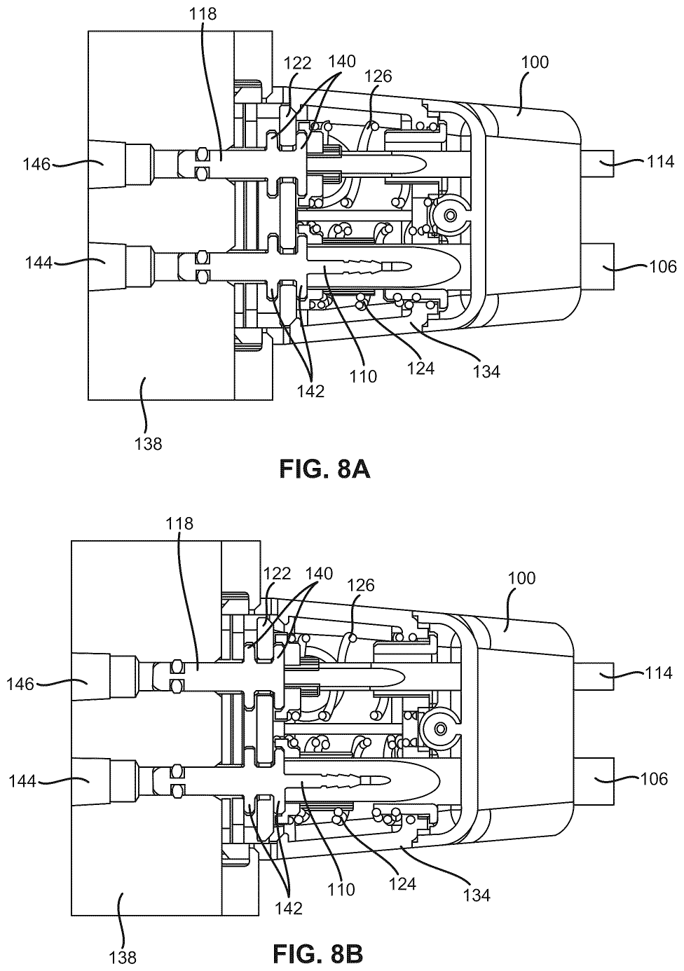

FIG. 8A illustrates the handle 100 in a locked configuration when coupled to the receptacle 138. The springs 124, 126 around each of the exhaust hose 106 and the inlet hose 114 can be compressed such that the plate 122 moves towards the proximal end of the handle 100. When the handle 100 is connected to the receptacle, the motion of the plate 122 locks the handle 100 to the receptacle 138 such that there is no risk that the identification card 130 or other components within the handle can be loose as the tabs 134 are locked from squeezing.

As seen in FIGS. 8A and 8B, exhaust connector 110 and inlet connector 118 can comprise projections 140, 142 in which the plate 122 rests within. The coupling between the connectors 110, 118 and the plate 122 are arranged such that when the connectors 110, 118 are connected to their receptive connectors in the receptacle 138, the plate 122 pushes away from receptacle 138 and compresses the spring, locking the handle 100. The connectors can move axially with the plate 122 during transition between the locked and unlocked configurations.

Upon locking, the separate connections of the exhaust hose 106, the inlet hose 114, and other connections (e.g., electrical connections for one or more thermocouples or thermistors) are confirmed when the tabs 134 lock into the receptacle 138 such that the user does not need to verify their connections. The exhaust hose 106 can fluidly couple with an exhaust lumen 144 in the receptacle. The inlet hose 114 can fluidly couple with an inlet lumen 146 in the receptacle. The exhaust lumen 144 and the inlet lumen 146 can extend through the probe.

The handle 100 maintains its locked configuration when the pressure of the inlet hose 114 or exhaust hose 106 is at or above a predetermined threshold in order to prevent unsafe unlocking of the handle 100 to the receptacle. The pressure can be determined by a pressure sensor incorporated into the handle 100 or alternatively, coupled to the card socket alongside the identification card 130. Once the pressure within the device 10 reaches a safe level for removal, the locking mechanism via the pressure plate 122 can be released such that the springs 124, 126 are relaxed, allowing the user to disconnect the handle 100. In some variations, the operating pressure of the device can be between about 750 psi and about 1500 psi.

In other variations, a ramp can be provided to push the inlet connector 118 forward to allow the handle 100 to be released. If the inlet connector 118 is under pressure, the user will not be able to overcome the pressure. This variation can be used without a plate or a spring, reducing the number of components and complexity.

FIG. 8B illustrates the handle 100 in an unlocked configuration when coupled to the receptacle 138. In this configuration, the springs 124, 126 around each of the exhaust hose 106 and the inlet hose 114 can be released such that the plate 122 is moved towards the distal end of the handle 100. The user can accordingly press the unlocked tabs 134 inward, unlocking the tabs 134 from the receptacle 138 and releasing the handle 100 accordingly.

Once released, the handle 100 can be removed from the receptacle 138 such that the identification card 130 can be removed from the card socket 128. The device 10 can be gamma sterilized more than once. In such cases, the identification card 130 can be removed to use a new card to ensure that gamma levels for the identification card 130 are not exceeded. The identification card 130 can be removed from the handle 100 and then sterilized with the device 10 to avoid damage to the identification card 130 due to radiation exposure.

In another variation, the identification card 130 can be replaced after the handle 100 is removed from receptacle 138 and connectors 110 and 118 are pushed into the handle 100, mimicking gas pressure. The identification card 130 can be pulled out and be replaced with a new card during this process. After connectors 110 and 118 are released, an aluminum plate lock (not shown) holds the identification card 130 in place when there is no gas pressure.

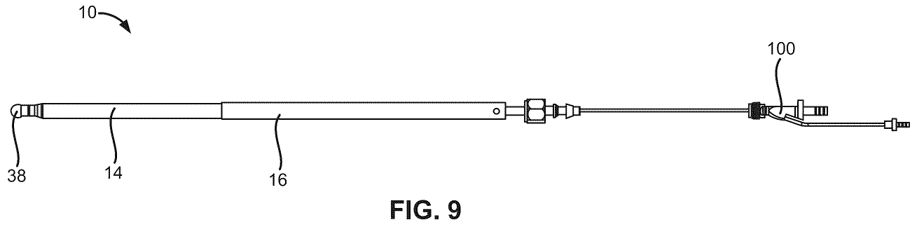

FIG. 9 illustrates a side view of a cryogenic device 10 for use with the handle 100. In this variation, the end effector 14 can comprise a rounded distal tip 38. The one or more passageways (e.g., the inlet lumen and the exhaust lumen) can extend through the end effector 14 beyond a proximal end of the handle.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations can be provided, or steps or operations can be eliminated or omitted from the described methods or processes to achieve the desired results. Moreover, any components or parts of any apparatus or systems described in this disclosure or depicted in the figures can be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings can be regarded in an illustrative rather than a restrictive sense.

Each of the individual variations or embodiments described and illustrated herein has discrete components and features which can be readily separated from or combined with the features of any of the other variations or embodiments. Modifications can be made to adapt a particular situation, material, composition of matter, process, process act(s) or step(s) to the objective(s), spirit, or scope of the present invention.

Methods recited herein can be carried out in any order of the recited events that is logically possible, as well as the recited order of events. Moreover, additional steps or operations can be provided or steps or operations can be eliminated to achieve the desired result.

Furthermore, where a range of values is provided, every intervening value between the upper and lower limit of that range and any other stated or intervening value in that stated range is encompassed within the invention. Also, any optional feature of the inventive variations described can be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter can conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein can be embodied in a non-transitory readable medium, machine-readable medium, and/or a machine accessible medium comprising instructions compatible, readable, and/or executable by a processor or server processor of a machine, device, or computing device. The structures and modules in the figures can be shown as distinct and communicating with only a few specific structures and not others. The structures can be merged with each other, can perform overlapping functions, and can communicate with other structures not shown to be connected in the figures. Accordingly, the specification and/or drawings can be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that can become obvious to those skilled in the art in view of this disclosure.

1. A cryogenic device for ablating tissue, comprising:
a handle comprising one or more tabs on the handle, wherein the handle comprises an unlocked configuration in which the one or more tabs are configured to be pressed into an interior of the handle;
an elongated probe extending from the handle, the elongated probe having a proximal end and a distal end, the distal end defining an end effector;
one or more fluid supply passageways extending within the elongated probe from the handle; and
wherein the handle comprises a locked configuration, wherein the one or more tabs are in a first position in the locked configuration and a second position in the unlocked configuration, wherein the one or more fluid supply passageways is pressurized when the handle is in the locked configuration.

2. The cryogenic device of claim 1, wherein the one or more fluid supply passageways comprises an exhaust passageway and an inlet passageway.

3. The cryogenic device of claim 1, further comprising an identification card within the handle.

4. The cryogenic device of claim 3, wherein the identification card is a FRAM card.

5. The cryogenic device of claim 3, wherein the identification card is replaceable.

6. The cryogenic device of claim 1, further comprising a receptacle configured to be locked to the handle.

7. The cryogenic device of claim 6, wherein the receptacle is configured to be unlocked from the handle upon actuation of a locking tab positioned on the handle.

8. The cryogenic device of claim 1, further comprising one or more electrical connections coupled to the handle.

9. The cryogenic device of claim 8, wherein the one or more electrical connections are each connected to a thermocouple.

10. The cryogenic device of claim 1, wherein the one or more tabs release to the unlocked configuration when pressure within the one or more fluid supply passageways is reduced to a predetermined threshold.

11. The cryogenic device of claim 1, further comprising one or more pressure sensors coupled to the handle.

12. A method for ablating tissue with a cryogenic device, the method comprising:
connecting a handle to a receptacle coupled to an elongated probe, wherein the handle comprises one or more tabs on the handle;
pressurizing one or more fluid supply passageways extending within the elongated probe from the handle;
delivering fluid to a distal end of the elongated probe;
ablating at least a portion of the tissue in contact with the distal end of the elongated probe; and
unlocking the handle by pressing the one or more tabs from a first position defining a locked configuration to a second position defining an unlocked configuration, wherein the one or more fluid supply passageways is pressurized when the handle is in the locked configuration, wherein the one or more tabs are pressed into an interior of the handle in the unlocked configuration.

13. The method of claim 12, wherein the one or more fluid supply passageways comprises an exhaust passageway and an inlet passageway.

14. The method of claim 12, further comprising an identification card within the handle.

15. The method of claim 14, wherein the identification card is a FRAM card.

16. The method of claim 14, wherein the identification card is replaceable.

17. The method of claim 12, further comprising a receptacle configured to be locked to the handle in the locked configuration.

18. The method of claim 17, wherein the receptacle is configured to be unlocked from the handle upon actuation of a locking tab positioned on the handle.

19. The method of claim 12, further comprising one or more electrical connections coupled to the handle.

20. The method of claim 19, wherein the one or more electrical connections are each connected to a thermocouple.

21. The method of claim 12, wherein the one or more tabs release to the unlocked configuration when pressure within the one or more fluid supply passageways is reduced to a predetermined threshold.

22. The method of claim 12, further comprising one or more pressure sensors coupled to the handle.
